SEQUENCE LISTING (8 sequences)

SEQ ID NO: 1 - DNA, 20 bp, Unknown
  NCBI_NM_011426.3
  prim_transcript (1)..(20)
aagtgtgctgtatgccccag

SEQ ID NO: 2 - DNA, 20 bp, Unknown
  NCBI_NM_011426.3
  prim_transcript (1)..(20)
ggaacagagacaggtgagcc

SEQ ID NO: 3 - DNA, 20 bp, Unknown
  NCBI_NM_000609.6
  prim_transcript (1)..(20)
tcgacgtctaagctgtgact

SEQ ID NO: 4 - DNA, 20 bp, Unknown
  NCBI_NM_000609.6
  prim_transcript (1)..(20)
ccatgtgtaggtgagctggg

SEQ ID NO: 5 - DNA, 18 bp, Unknown
  NCBI_NM_000609.6
  prim_transcript (1)..(18)
atgcccatgccgattctt

SEQ ID NO: 6 - DNA, 20 bp, Unknown
  NCBI_NM_000609.6
  prim_transcript (1)..(20)
gccgggctacaatctgaagg

SEQ ID NO: 7 - DNA, 20 bp, Unknown
  NCBI_NM_020530.1
  prim_transcript (1)..(20)
ggggtactgctcacacagag

SEQ ID NO: 8 - DNA, 24 bp, Unknown
  NCBI_NM_020530.1
  prim_transcript (1)..(24)
tacgtatataggggtccaggagtc

(19) United States
(12) Patent Application Publication
(10) Pub. No.: US 2017/0327573 A1
(43) Pub. Date: Nov. 16, 2017

(54) Composition to induce bone marrow stem cell mobilization

(71) Applicant: Universitá degli Studi di Padova, Padova (IT)

(72) Inventors: Gian Paolo Fadini, Albignasego (PD) (IT); Mattia Albiero, Torri di Quartesolo (VI) (IT); Stefano Ciciliot, Montegrotto Terme (PD) (IT)

(21) Appl. No.: 15/519,091

(22) PCT Filed: Sep. 22, 2015
(86) PCT No.: PCT/IB2015/057285

§ 371 Date: Apr. 13, 2017

(30) Foreign Application Priority Data
Sep. 24, 2014 (IT) GE2014A000095

(51) Int. Cl. C07K 16/24; A61K 39/395; A61K 45/06; C07K 16/28; A61K 39/00

(52) U.S. Cl. CPC C07K 16/248; A61K 39/3955; C07K 16/2866; A61K 39/39541; A61K 45/06; C07K 2317/76; A61K 2039/505

(57) ABSTRACT

A pharmaceutical composition to induce bone marrow stem cell mobilization from the bone marrow to peripheral blood in patients suffering from pathological conditions, such as diabetes, or subjected to treatments that impair cell mobilization, or in patients suffering from the so called “poor mobilizer” condition, includes at least one therapeutic agent that inhibits production and/or action of the human cytokine oncostatin M (OSM), a macrophage derived factor, that prevents mobilization of stem cells.

### FIELD OF THE INVENTION

The present invention relates to a composition to induce mobilization or migration of bone marrow stem cells, that is not embryonic stem cells, from the bone marrow to the peripheral blood.

### INCORPORATION BY REFERENCE OF MATERIAL SUBMITTED ON A COMPACT DISK OR AS A TEXT FILE VIA THE OFFICE ELECTRONIC FILING SYSTEM (EFS-WEB)

The present specification incorporates by reference the material in ASCII text file “SeqListing2_ST25” created on Jul. 21, 2017 and having a size of 2097 bytes.

### BACKGROUND OF THE INVENTION

Diabetes mellitus is a chronic disease characterized by a reduction in life expectancy, due to the development of serious multiorgan complications.

The risk of cardiovascular events is up to four times higher in diabetic patients than in non diabetic patients.

60% of diabetes-related excess mortality depends on cardiovascular causes, but diabetes increases also the risk of death from cancers, infectious diseases and degenerative disorders (Seshasai, 2011).

The mechanisms determining excess vascular damage in diabetes are not completely known.

Due also to such reason, the therapies known in the prior art aiming at preventing cardiovascular events in patients suffering from diabetes are ineffective or insufficient (Pillarisetti, 2012).

It is believed that a reduction and dysfunction of bone marrow-deriving stem cells contributes to tissue damage in diabetes (Fadini, 2014).

Recently it has been proved that diabetes impairs stem cell mobilization from the bone marrow to the peripheral blood that can be obtained in response to tissue ischemia and to administration of growth factors (such as G-CSF) (Fadini, 2006; Ling, 2012; Fadini, 2013).

This is true both in animal experimental models, and for human beings.

In the hematological context diabetes has been recently enumerated among the so called “poor mobilizer” conditions, since it increases the risk of failure of a cycle of mobilization therapy with G-CSF (Granulocyte colony-stimulating factor) and/or chemotherapy for stem cell auto-transplantation (Ferraro, 2011).

The treatment of the “poor mobilizer” condition is very limited and it requires the use of expensive drugs with a limited efficacy over time, such as the reversible and selective antagonist of CXCR4 chemokine receptor (AMD3100 or Plerixafor), that acts by determining the block of the binding of its cognate ligand, the stromal cell derived factor-1α (SDF-1α) (Bakanay, 2012).

The extent of bone marrow stem cell mobilization directly influences and predicts the engraftment after auto-transplantation of the stem cells, and therefore it affects hematopoietic recovery, morbidity and mortality of patients after the transplantation (D'Rozario, 2014).

Moreover the level of circulating bone marrow-derived stem cells is a predictor of the risk of developing future cardiovascular events, such as heart attack, stroke, heart failure (Fadini, 2010).

The degree of mobilization of bone marrow stem cells after acute myocardial infarction predicts the development of left ventricular dysfunction (Leone, 2005), and the future development of further adverse cardiovascular events (Ling, Shen et al., 2012).

Finally bone marrow stem cell mobilization, combined or not with the auto-transplantation of the cells themselves at the level of ischemic organs, has been used for treating cardiovascular diseases, such as myocardial infarction (Zimmet, 2012; Moazzami, 2013) and critical lower limb ischemia (Fadini, 2010).

Therefore the defect of responsivity of bone marrow to the mobilization of stem cells may have adverse implications for diabetic patients both as regards the development of cardiovascular complications and as regards the efficacy and engraftment of the bone marrow transplantation.

The mechanisms underlying the mobilization defect of stem cells in diabetes are not completely known and some researchers are studying diabetes as a model for other “poor mobilizer” conditions (Ferraro, Lymperi et al., 2011).

It is believed that a change in the regulation of SDF-1α (Stromal Derived factor 1-alfa, called also as CXCL12), plays a crucial role, since CXCL12/SDF-1α is one of the molecules more involved in the traffic of stem cells from and to the bone marrow (Lapidot, 2002).

CXCL12/SDF-1α is a chemokine that acts by binding to its own CXCR4 receptor expressed on the surface of hematopoietic stem cells. High intra-marrow concentrations of CXCL12/SDF-1α lead to retention of stem cells, while a reduction of intra-marrow concentrations of CXCL12/SDF-la and/or an increase of concentrations of CXCL12/SDF-1α in peripheral blood lead to mobilization of stem cells. A defect in the regulation of intra-marrow concentrations of CXCL12/SDF-1α in diabetes and in other “poor mobilizer” conditions prevents stem cells from mobilizing.

Under basal conditions, a high intra-marrow concentration of CXCL12/SDF-la chemokine retains hematopoietic stem cells in the bone marrow, while in response to the stimulation with biological agents or mobilizing conditions, the concentration of CXCL12/SDF-la is reduced (Levesque, 2003).

CXCL12/SDF-1α is secreted mainly by mesenchymal stem/stromal cells and it is stimulated in response to an unknown soluble factor produced by macrophages that express the CD169 adhesion molecule (Chow, 2011).

In response to G-CSF factor, mesenchymal stromal cells of bone marrow reduce their expression and production of CXCL12/SDF-la, promoting the mobilization of stem cells from the bone marrow to peripheral blood (Levesque, Hendy et al., 2003).

The mechanism by means of which G-CSF acts on mesenchymal cells is not completely known, but it is believed that it suppresses macrophage activity, by removing the restraint imposed to the mobilization by stromal-derived CXCL12/SDF-la (Chow, Lucas et al., 2011).

### SUMMARY OF THE INVENTION

The object of the present invention is to identify a macrophage derived factor able to stimulate the production of CXCL12/SDF-1α by stromal cells and to inhibit said macrophage-derived factor such to stimulate the migration or mobilization of marrow stem cells from the bone marrow to the peripheral blood. Such increase of circulating stem cells can be used for making transplantations of marrow stem cells and/or for promoting endogenous protection against cardiovascular diseases.

The object of the present invention is to develop a pharmaceutical composition and a method easy to be used and inexpensive permitting the mobilization of marrow stem cells.

A further object of the present invention is to develop a method and a kit for helping in the diagnosis and that can be used for helping in identifying “poor mobilizer” conditions, together with the clinical history of the patient and with other possible diagnostic tests subjected to the evaluation by a qualified pathologist.

According to the invention the main object is achieved by a pharmaceutical composition to induce bone marrow stem cell mobilization from the bone marrow to peripheral blood in patients suffering from pathological conditions, such as diabetes, or subjected to treatments that impair cell mobilization, or in patients suffering from the so called “poor mobilizer” condition, which composition comprises at least one therapeutic agent that inhibits production and/or action of the human cytokine oncostatin M (OSM), a macrophage derived factor such as a OSM blocking monoclonal antibody or receptor inhibitor, that prevent mobilization of stem cells.

Oncostatin M is a known 28-kDa glycoprotein of cytokine family (such as described for example in Kishimoto T. et al. (1995) Blood 86: 1243-1254).

According to the present invention said therapeutic agent or agents carry out one or more of the following functions, provided in combination or alternatively to one another:

a) inhibition of oncostatin M (OSM) production by cells;

b) neutralization and/or blocking of extracellular oncostatin M (OSM);

c) inhibition and/or neutralization of oncostatin M (OSM) receptor or a subunit thereof;

d) inhibition of the binding of oncostatin M (OSM) to its receptor;

e) inhibition and/or blocking of intracellular effects of the activation of the receptor by oncostatin M (OSM).

In the functions mentioned above, said oncostatin M (OSM) receptor can be OSMR (oncostatin M receptor) or gp130 (interleukin-6 family receptor) or OSMR/gp130 heterodimer.

The present invention intends to overcome the limits existing in prior art in the field of systems, treatments and processes able to promote mobilization of marrow stem cells in patients suffering from diabetes or suffering from “poor mobilizer” condition, for auto-transplantation of bone marrow or for cardiovascular protection.

Said composition can be used for:

mobilizing marrow stem cells in patients that will have to be subjected to auto-transplantation of marrow stem cells collected by apheresis from peripheral blood for treatment of hematologic or cardiovascular diseases,

mobilizing marrow stem cells for prevention or treatment of acute cardiovascular diseases, for example stroke, myocardial infarction, acute lower limb ischemia, or chronic diseases for example heart failure, cardiac ischemia, lower limb obliterative arteriopathy,

mobilizing marrow stem cells from donors, to be collected by apheresis from peripheral blood.

As described in more details below, the invention relates also to a kit and a method for helping in the diagnosis of conditions of inflammation and/or of impaired or absent marrow stem cell mobilization from the bone marrow to peripheral blood comprising the step of evaluating the gene and/or protein expression of CD169 (called also as sialoadhesin or SIGLEC-1), CD169 being a adhesion molecule expressed on the plasma membrane of macrophages that produce the cytokine oncostatin M (OSM).

The present invention relates also to a method for the prevention or the treatment of patients affected by diabetes and/or cardiovascular diseases and/or patients suffering from impaired or absent marrow stem cell mobilization from the bone marrow to peripheral blood, so-called “poor mobilizer”, characterized in that it comprises one or more of the following steps:

a) stimulation of the mobilization of marrow stem cells or progenitor cells from the bone marrow to peripheral blood;

b) collection of said cells from peripheral blood through apheresis or the like;

c) administration of said cells to the patient through infusion and/or injection and/or transplantation,

said step of stimulation being achieved through the administration of a composition comprising at least one therapeutic agent that inhibits the production and/or the action of the human cytokine oncostatin M (OSM).

These and other characteristics and advantages of the present invention will be more clear from the following description of some embodiments shown in the annexed drawings wherein:

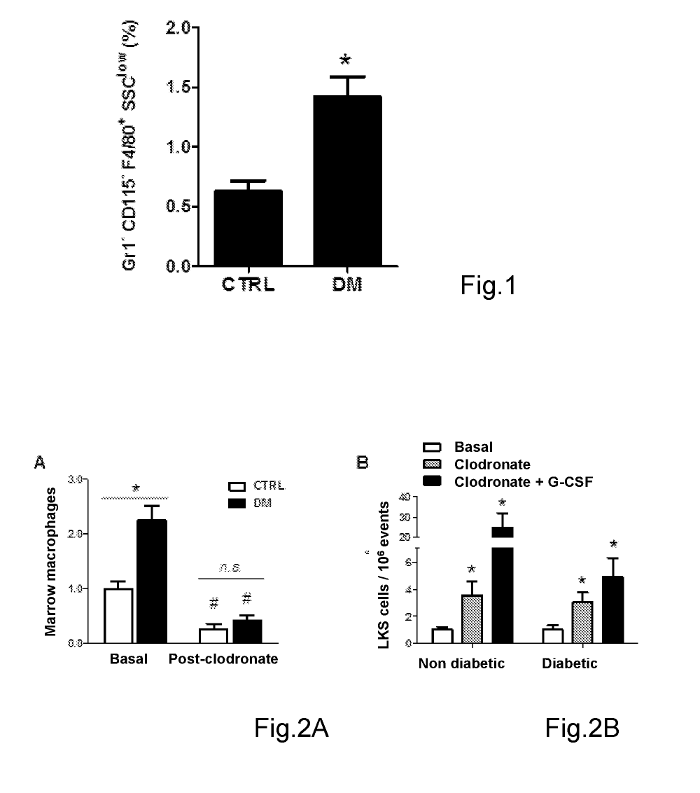

FIG. 1 shows the percentage of macrophages with respect to total marrow cellularity in diabetic mice (DM) and non diabetic control mice (CTRL). (*p<0.05 in diabetic mice (DM) versus non diabetic mice (CTRL));

FIG. 2A shows that the injection of clodronate-containing liposomes (post-clodronate) with respect to basal (Baseline) significantly reduces the percentage of marrow macrophages in diabetic mice (DM) and non diabetic mice (CTRL) (*p<0.05 in diabetic mice (DM) versus non diabetic mice (CTRL)); #p<0.05 post-clodronate versus basal; n.s.: not statistically significant;

FIG. 2B shows that the administration of clodronate-containing liposomes increases the level of circulating stem cells negative for lineage markers (Lin−) and that express Sca-1 and c-Kit (also of LKS cells) with respect to basal one (Baseline), with or without subsequent administration of G-CSF (*p<0.05 versus basal);

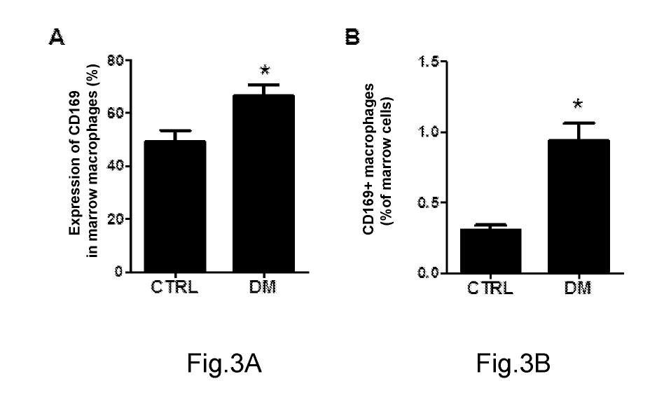

FIG. 3A shows the percentage of surface expression of CD196 on marrow macrophages in diabetic mice (DM) and control mice (CTRL) (*p<0.05 in diabetic mice (DM) versus control mice (CTRL));

FIG. 3B shows the percentage of CD169-expressing macrophages with respect to total marrow cellularity in diabetic mice (DM) and control mice (CTRL) (*p<0.05 in diabetic mice (DM) versus control mice (CTRL));

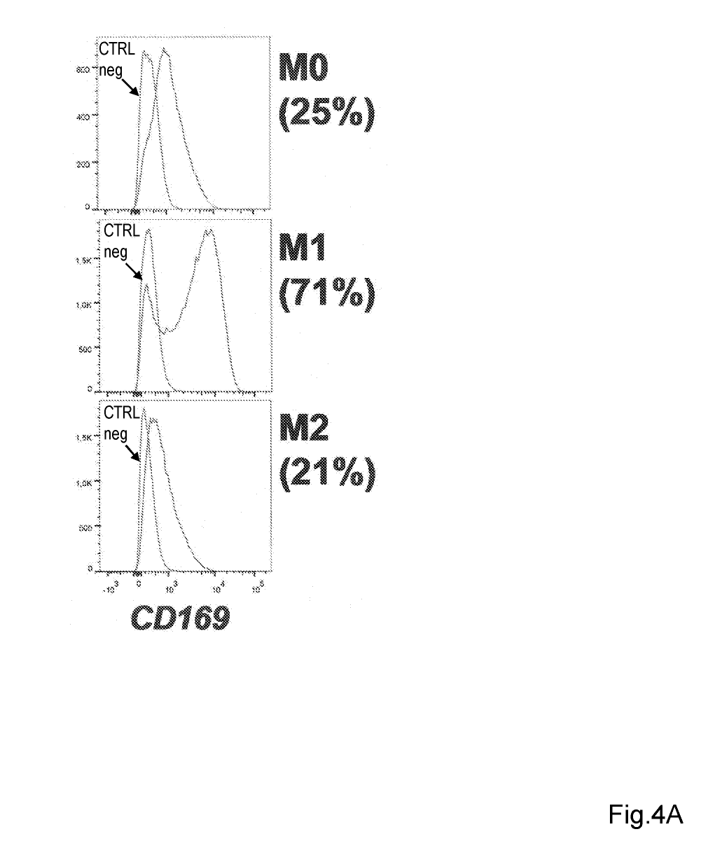

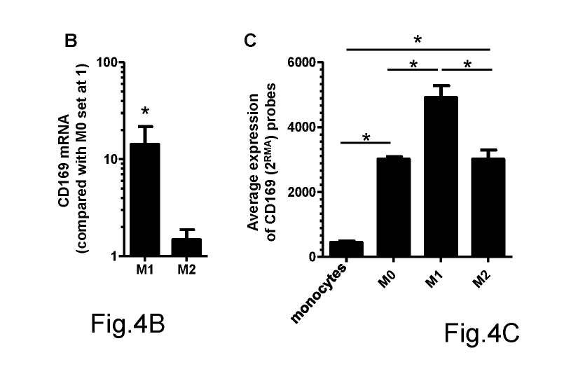

FIG. 4A shows the surface expression of CD169 with respect to the negative control (neg CTRL), evaluated by flow cytometry, in M0, M1 and M2 human macrophages in vitro;

FIG. 4B shows the gene expression, evaluated by qPCR, of CD169 in M1 and M2 macrophages (with respect to M0 set to 1) (*p<0.05 in M1 versus M2);

FIG. 4C shows “in silico” analysis of the expression of <219519_s_at> and <44673_at> probes, specific for CD169, in monocytes, M0, M1 and M2 macrophages of the GEO dataset GDS2429 (*p<0.05 in shown comparisons);

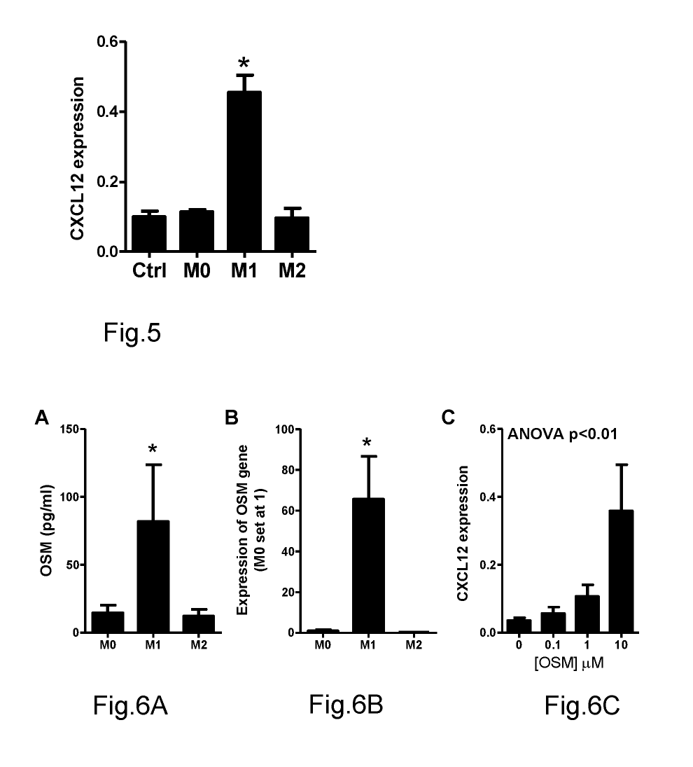

FIG. 5 shows the effect of human M0, M1 and M2 macrophages-conditioned media on gene expression, evaluated by qPCR, of SDF-la/CXCL12 in human mesenchymal cells, compared with control condition (culture without conditioned medium), (*p<0.05 compared to control condition (Ctrl));

FIGS. 6A, 6B and 6C respectively show the protein contents of oncostatin M (OSM), evaluated by ELISA assay, in human M0, M1 and M2 macrophage-conditioned media (panel A, *p<0.05 in M1 compared with M0 and M2); the gene expression of oncostatin M (OSM), evaluated by qPCR, in human M0, M1 and M2 macrophages (panel B, *p<0.05 in M1 compared with M0 and M2); the effect of increasing concentrations of oncostatin M (OSM) on gene expression, evaluated by qPCR, of SDF-la/CXCL12 in human marrow mesenchymal cells (panel C) (ANOVA, analysis of variance);

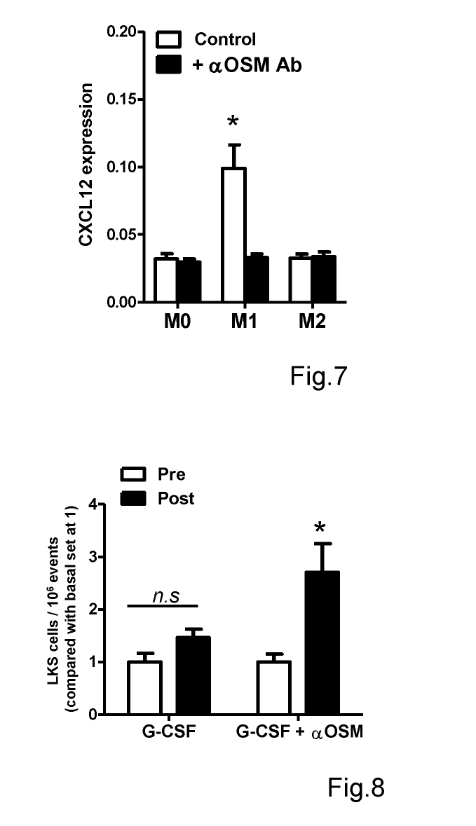

FIG. 7 shows the effect of neutralization of oncostatin M (OSM), by a polyclonal neutralizing antibody directed against the human OMS protein, on the capacity of human MO, M1 and M2 macrophage-conditioned media to induce gene expression of SDF-1α/CXCL12 in human marrow mesenchymal cells (*p<0.05 in the indicated condition compared with MO and M2);

FIG. 8 shows the effect of the neutralization of oncostatin M (OSM), by the injection of a neutralizing antibody directed against the murine (aOSM) oncostatin M (OSM) protein, on the mobilization of LKS stem cells induced by G-CSF in diabetic mice (*p<0.05 post versus pre-administration of the treatment shown on X axis; n. s. non statistically significant).

### DETAILED DESCRIPTION OF EMBODIMENTS OF THE INVENTION

In the present text the following acronyms are used:

CXCL12: C-X-C ligand-12;

SDF-1α: Stromal Derived Factor-1α;

CD: Cluster of Differentiation;

OSM: oncostatin M;

SSC: Side scatter;

IL: interleukin;

LPS: liypopolysaccharide;

IFN-γ: interferon gamma;

qPCR: quantitative polymerase chain reaction (also real-time PCR);

FACS: Fluorescence Activated Cell Sorting (flow cytometry);

LKS: Lineage negative, c-Kit positive, Sca-1 positive (cells negative for lineage markers, positive for Sca-1 and c-Kit);

G-CSF: granulocyte colony stimulating factor;

NCBI: National Center for Biotechnology Information.

Moreover in the text of the description and claims the term “poor mobilizer” means any condition occurring in a patient that leads to a defect of responsivity of bone marrow to stem cell mobilization.

As described better below the present invention relates also to a composition and method for promoting the marrow stem cell mobilization by inhibition of the production and/or action of the human cytokine oncostatin M (OSM), a macrophage derived factor abundantly expressed in patients under “poor mobilizer” condition.

Said factor physiologically inhibits the mobilization of marrow stem cells.

The mobilization of marrow stem cells allows cardiovascular diseases to be prevented or treated in a simple and efficacious manner.

Moreover the mobilization of marrow stem cells allows a transplantation or auto-transplantation of marrow stem cells collected by apheresis to be made in an efficacious manner for the patient.

Preferably the composition of the present invention permits the mobilization of marrow stem cells in patients suffering from diabetes or suffering from the so called “poor mobilizer” condition such to avoid or reduce cardiovascular complications, promoting endogenous protection and such to increase the success of bone marrow transplantations.

The experimental process that has led to identify the oncostatin M as the factor responsible for the inhibition of the mobilization of marrow stem cells is described below.

Said process comprises the following steps:

1. Determining the level of intra-marrow macrophages;

2. Evaluating the expression of CD169;

3. Determining the macrophage-derived soluble factor;

4. Validating the role of OSM in vitro;

5. Validating of the role of OSM in vivo.

1. Determining the Level of Intra-Marrow Macrophages

The level of intra-marrow macrophages in mice of the common strain called as C57B1/6J, induced with diabetes by injecting streptozotocin and in age-matched non diabetic control mice was determined by using flow cytometry.

The bone marrow obtained from animals by elution of cell contents of femurs and tibiae was analyzed 4 weeks after hyperglycemia development. Age-matched non diabetic C57B1/6J mice were used as the control.

Such as shown in FIG. 1 it was observed that the bone marrow of diabetic mice contains a percentage of macrophages significantly higher (more than twice) than the bone marrow of non diabetic mice. Such macrophages have Grl⁻CD115⁻F4/80⁺SSC^{low} phenotype, that is they express the typical murine macrophage marker F4/80, but not monocyte markers CD115 and Grl and have a low nuclear complexity.

In order to evaluate the pathogenetic meaning of the excess of intra-marrow macrophages in the diabetic mouse, a method has been used able to determine the selective death of macrophages in the reticuloendothelial system, by injecting clodronate-containing liposomes.

Due to their phagocytic activity, the macrophages actively ingest liposomes, promoting the reaching of high intracellular clodronate concentrations, that are toxic for the cell and causes its death (Van Rooijen 1994).

The injection of clodronate-containing liposomes has proved to be efficacious in suppressing the levels of marrow macrophages both in diabetic mice and in non diabetic mice, and it has been proved to be able to restore spontaneous or G-CSF (Granulocyte Colony-Stimulating Factor) induced mobilization of LKS stem cells, in diabetic mice, FIG. 2. Such LKS cells (Lin⁻Sca-1⁺c-Kit⁺), that do not express lineage markers (Lin″) and express Sca-1 and c-Kit, represent the typical hematopoietic marrow stem cells.

Therefore, it has been proved that the excess of marrow macrophages in the diabetes contributes in the mobilization defect.

2. Evaluating the Gene Expression of CD169

The expression of CD169 gene has been evaluated on the surface of the marrow macrophages Grl⁻CD115⁻ F4/80⁺SSC^{low} deriving from diabetic and non diabetic mice by flow cytometry analysis.

The gene expression of CD169 has been analysed in macrophages isolated by means of FACS (fluorescent-activated cell sorter) from diabetic and non diabetic mice by means of qPCR (primers forward AAGTGTGCTGTATGCCCCAG [SEQ ID NO. 1]; reverse GGAACAGAGACAGGTGAGCC [SEQ ID NO. 2]; reference sequence NCBI NM_011426.3),

As experimentally shown in FIG. 3, the surface protein expression of CD169 and the gene expression of CD169 resulted significantly increased in marrow macrophages of diabetic mice compared to non diabetic mice.

As a result, the diabetic mice have an excess of CD169⁺ macrophages that, according to the literature, provide an indication of retention of stem cells in the bone marrow and inhibit their mobilization in the peripheral circulation.

To characterize the type of macrophages where CD169 is selectively expressed, human monocytes deriving from peripheral blood of anonymous blood donors were cultured, differentiated into macrophages in vitro inducing polarization to MO (resting, non stimulated macrophages), M1 (macrophages stimulated with LPS (lipopolysaccharide) and IFN-γ) and M2 (macrophages stimulated with interleukin-4+interleukin-13) according to a standard protocol (Fadini, 2013) and flow cytometry and qPCR methods were used (primers forward TCGACGTCTAAGCTGTGACT [SEQ ID NO. 3]; reverse CCATGTGTAGGTGAGCTGGG [SEQ ID NO. 4]; reference sequence NCBI NM_023068.3) to determine the surface protein expression and gene expression of CD169.

CD169 is the result most significantly expressed in M1 macrophages compared to MO and M2 ones, both as regards surface protein expression, and as regards the gene expression, such as shown by the results of FIG. 4.

Moreover by the analysis of gene expression profiles (dataset GDS2429 submitted in public database http://www.ncbi.nlm.nih.gov/sites/GDSbrowser?acc=GDS242 9) of human monocytes and macrophages polarized in culture to M0, M1 and M2, it has been possible to prove that CD169 expression (<219519_s_at> and <44673_at> probes of HGU133A array) is significantly higher in proinflammatory M1 macrophages than in monocytes, non polarized M0 macrophages and anti-inflammatory M2 macrophages.

Therefore such data show that CD169 gene is a specific marker of human M1 macrophages in vitro.

3. Determining Macrophage-Derived Soluble Factor

Considering that the mobilization inhibiting activity is carried out by marrow macrophages expressing CD169 through a soluble factor not identified yet, conditioned media from cultures of human MO, M1 and M2 macrophages were obtained, carried out as mentioned above.

Therefore the effect of such media on the gene expression of SDF-1α/CXCL12 in human (stromal) mesenchymal stem cells by qPCR was evaluated (primers forward ATGCCCATGCCGATTCTT [SEQ ID NO. 5]; reverse GCCGGGCTACAATCTGAAGG [SEQ ID NO. 6]; reference sequence NCBI NM_000609.6).

It has been found that only the conditioned medium from human M1 macrophages, but not M0 and M2 macrophages, induces the expression of CXCL12/SDF-la by human marrow stromal cells, such as shown in FIG. 5.

Therefore it has been supposed that the macrophage-derived soluble factor carrying out the activity of retention of stem cells in the bone marrow is contained only in the conditioned medium from M1 macrophages, and not in that from M0 and M2 macrophages.

In order to identify a relatively restricted list of potential candidate factors for being said factor secreted in the M1 macrophage medium, a “in silico” analysis has been developed for the gene expression profiles of human and murine M0, M1 and M2 macrophages, to search for secreted gene products, differentially expressed by M1 macrophages with respect to M0 and M2 macrophages, and that have a receptor expressed in mesenchymal stromal cells.

To this end human and murine macrophage microarray data submitted in public databases have been used (series GSE5099 and GSE32690 respectively), an analysis of differential gene expression M1 vs M0 and M1 vs M2 has been performed with stringent criteria (p<0.001 and fold change>5), and the results have been cross-checked with a secreted protein database (Secreted Protein Database, http://spd.cbi.pku.edu.cn) and with a database of gene expression of human and murine marrow mesenchymal cells (series GSE6029 and GSE43781 respectively).

The candidate factors have been further arranged on the basis of a systematic search of scientific literature (Pubmed search key: (“CXCL12” OR “SDF”) AND “mesenchymal”) to identify soluble factors that potentially may induce expression of CXCL12/SDF-la in mesenchymal cells. By such “data mining” methodological approach, it has been possible to identify oncostatin M (OSM), a possible mediator of the effect of M1 macrophages on gene expression of CXCL12 by stromal mesenchymal cells.

Studies already present in scientific literature have separately indicated that oncostatin M can be able to induce expression of SDF-la/CXCL12 in mesenchymal-derived cells (Lee, 2007), that oncostatin M is produced preferentially by pro-inflammatory macrophages (Guihard, 2012), and that it can play a role in the regulation of the hematopoietic system (Minehata, 2006).

4. Validating the Role of OSM In Vitro;

In order to validate the role of oncostatin M (OSM) in the regulation of the mobilization of marrow hematopoietic stem cells, concentrations of oncostatin M (OSM) were determined in conditioned media from human M0, M1 and M2 macrophages by ELISA assay (ELH-OSM, RayBiotech, Inc.).

Oncostatin M (OSM) resulted to be more concentrated in conditioned media from M1 macrophages than M0 and M2. Moreover the gene expression of oncostatin M (OMS) evaluated by qPCR (primers: forward GGGGTACTGCTCACACAGAG [SEQ ID NO. 7]; reverse TACGTATATAGGGGTCCAGGAGTC [SEQ ID NO. 8]; reference sequence NCBI NM_020530.4) resulted to be more expressed (>60 times) in M1 macrophages, than M0 and M2 such as shown in FIG. 6.

Therefore the capacity of oncostatin M (OSM) to induce expression of SDF-la/CXCL12 in human mesenchymal stem cells has been determined by qPCR and it has been found that increasing concentrations of oncostatin M (OSM) progressively increase the expression of SDF-la/CXCL12.

Finally, in order to evaluate whether oncostatin M (OSM) is the soluble factor in the M1 conditioned medium that increases the expression of SDF-1α/CXCL12 in human mesenchymal stem cells, such cells were cultured in presence and absence of M0, M1, M2 conditioned media and in presence and absence of an anti-human OMS neutralizing monoclonal antibody (MAB295, R&D Systems). This led to demonstrate that the capacity of the M1 macrophage conditioned medium to induce expression of CXCL12/SDF-1α in marrow stromal cells is completely suppressed by the neutralization of oncostatin M (OSM) (FIG. 7).

Therefore, in vitro, oncostatin M (OSM) is the soluble factor produced by M1 macrophages that increases the expression of SDF-1α/CXCL12 in marrow stromal cells, preventing hematopoietic stem cells from being mobilized from the bone marrow to the periphery.

5. Validating the Role of OSM In Vivo.

In order to obtain in vivo validation of such mechanism, inhibition of oncostatin M (OSM) has been carried out in mice induced with diabetes by injecting streptozotocin as mentioned above and analyzed after 4 weeks from hyperglycemia development, by intraperitoneal injection of a polyclonal neutralizing antibody (AF-495-NA, R&D Systems) specific for murine oncostatin M (OSM), before beginning a marrow stimulation cycle by a subcutaneous injection of G-CSF (200 g/kg/die for 4 consecutive days).

The results show that, while the administration of only G-CSF is not able to induce mobilization of LKS stem cells in diabetic mice, the inhibition of oncostatin M (OMS) in vivo by the neutralizing antibody recovers the mobilization in response to G-CSF in diabetic mice (FIG. 8).

From such experimental data it is clear that oncostatin M is the macrophage derived factor that inhibits mobilization of stem cells from the bone marrow to peripheral blood: the inhibition of oncostatin M permits the mobilization of marrow stem cells in “poor mobilizer” conditions and particularly in patients suffering from diabetes.

Therefore the present invention relates to a pharmaceutical composition to induce marrow stem cell mobilization from the bone marrow to peripheral blood in patients suffering from pathological conditions, such as diabetes, or subjected to treatments that impair cell mobilization, or suffering from the so called “poor mobilizer” condition, which composition comprises at least one therapeutic agent that inhibits production and/or action of the human cytokine oncostatin M (OSM), a macrophage derived factor that prevents mobilization of stem cells.

As it is known the bone marrow is the main source for hematopoietic stem cells and of mesenchymal stem cells.

Therefore the term marrow stem cells means: hematopoietic stem cells, hematopoietic progenitor cells, endothelial progenitor cells.

Therefore at least one inhibitor of the production and/or action of the oncostatin M (OSM) can be used for making a medicament to induce marrow stem cell mobilization from the bone marrow to the peripheral blood in patients with impairment of cell mobilization, so called “poor mobilizer” condition and/or suffering from cardiovascular diseases.

Said therapeutic agent or agents carry out one or more of the following functions, provided in combination or alternatively with each other:

a) inhibition of oncostatin M (OSM) production by cells;

b) neutralization and/or blocking of extracellular oncostatin M (OSM);

c) inhibition and/or neutralization of oncostatin M (OSM) receptor or a subunit thereof;

d) inhibition of the binding of oncostatin M (OSM) to its receptor;

e) inhibition and/or blocking of intracellular effects of the activation of the receptor by oncostatin M (OSM).

According to the preferred embodiment, said therapeutic agent is a monoclonal antibody specifically directed against human oncostatin M. Such antibody is able to neutralize the biological activity of the oncostatin M (OMS) in extracellular fluids, preventing oncostatin M from binding to its receptor.

Such preferred embodiment derives directly from the example shown in FIG. 8, according to which the intraperitoneal injection of a murine anti-OSM neutralizing antibody is able to recover the mobilization of marrow stem cells in addition to the treatment by G-CSF.

For example said therapeutic agent can be the GSK315234 monoclonal antibody that is the humanized anti-OSM immunoglobulin G1 (IgG1) monoclonal antibody.

As an alternative or in combination said therapeutic agent is at least one monoclonal antibody specifically directed against oncostatin M receptor or a subunit of said receptor.

According to one embodiment said therapeutic agent is at least a monoclonal antibody specifically directed against OSMR (Oncostatin M receptor) or gp130 (interleukin-6 family receptor) or the OSMR/gp130 heterodimer. Said at least one therapeutic agent can be a binding protein, a soluble receptor, a degrading enzyme, a neutralizing antibody, a blocking monoclonal antibody or a fragment thereof, said compounds being able to inhibit or neutralize the oncostatin M protein, by sequestering and/or degrading oncostatin M and/or preventing it from binding to its receptor and said compounds being comprised in the pharmaceutical composition alone or in a mixture with one another.

According to one embodiment the composition comprises, as therapeutic agent, single-stranded R A synthetic oligonucleotides (antisense RNA) and/or double-stranded RNA such as siRNA (small interfering RNA), complementary to mRNA (messenger RNA) encoding for oncostatin M, said gene silencers being comprised in combination or alternatively to one another such to obtain silencing, that is the decreased expression of gene encoding for oncostatin M.

According to a further embodiment the composition comprises, as therapeutic agent, single-stranded RNA synthetic oligonucleotides (antisense RNA) and/or double-stranded RNA, such as siRNA (small interfering RNA), complementary to mRNA (messenger RNA) encoding for oncostatin M receptor or a subunit of said receptor, said gene silencers being comprised in combination or alternatively to one another, such to obtain silencing, that is the decreased expression of gene encoding for the oncostatin M receptor or a subunit of said receptor.

Said at least one therapeutic agent can be a compound or a mixture of compounds able to inhibit oncostatin M production at cellular level, such as an enzyme inducer, an enzyme or receptor inhibitor, a ligand of a receptor in the cell surface or cytoplasm or nucleus, a compound that is toxic for the cells that produce oncostatin M (OSM), an antisense RNA, said compounds being comprised in the pharmaceutical composition alone or in a mixture with one another.

Said at least one therapeutic agent can be a compound or a mixture of compounds able to inhibit or neutralize oncostatin M receptor or a subunit of said receptor, such as a chemical inhibitor or antagonist or partial agonist of said receptor, a degrading enzyme, an antibody blocking said receptor, a monoclonal antibody blocking said receptor, a fragment of a monoclonal antibody directed against said receptor, an antisense RNA directed against the messenger RNA of the receptor gene or any agent that prevents oncostatin M from eliciting its biological effects through the binding to its receptor, said compounds being comprised in the pharmaceutical composition alone or in a mixture with one another.

According to a further embodiment at least one therapeutic agent is a compound or a mixture of compounds able to inhibit the biological effects of oncostatin M in target cells or tissues, such as a chemical compound that targets a molecule that is part of the intracellular transduction signalling cascade elicited by the binding of oncostatin M to its receptor, a chemical compound which is a receptor or enzyme inducer or inhibitor, a ligand of a receptor in the cell surface, and/or cytoplasm and/or nucleus, an antisense RNA, said compounds being comprised in the pharmaceutical composition alone or in a mixture with one another.

The administration of said therapeutic agent or agents can occur through carriers able to optimize delivery to the target organ or cell, that is bone marrow and stem cell niche, such as liposomes, exosomes, micelles, microparticles, nanoparticles, nanostructured carriers, said carriers being provided alone or combined with each other.

Moreover said at least one therapeutic agent can be provided in combination with one or more chemotherapies and/or growth factors, such as G-CSF, GM-CSF (granulocyte-macrophage colony-stimulating factor) or the like, and/or chemokines (for example SDF-1α/CXCL12).

Said at least one therapeutic agent is provided in combination with at least one pharmacologically acceptable excipient for the treatment of pathological conditions of impaired cell mobilization from the bone marrow to peripheral blood, so-called “poor mobilizer” condition, and/or the treatment of phenomena associated thereto, and/or the treatment of cardiovascular diseases.

According to the present invention in order to inhibit the production and/or action of cytokine oncostatin M it is possible to use:

one or more monoclonal antibodies such as those described in the U.S. Pat. No. 5,907,033 or EP 451612;

one or more compounds such as those described in patent application WO 1999/48523;

at least one soluble receptor able to bind oncostatin M, such as described in U.S. Pat. No. 5,783,672;

one or more oncostatin M protein mutant analogs such as described in U.S. Pat. No. 5,874,536;

one or more oncostatin M antigen binding proteins, such as described in patent EP 2643352.

Said pharmaceutical composition can be used for first use in a medical treatment of patients suffering from cardiovascular diseases and/or pathological conditions, such as diabetes, and/or subjected to treatments that impair cell mobilization, i.e. patients affected by the so-called “poor mobilizer” condition.

Said pharmaceutical composition can be used for the implementation of a medicament for the prevention or the treatment of patients suffering from cardiovascular diseases and/or pathological conditions, such as diabetes, and/or subjected to treatments that impair cell mobilization, that is patients affected by the so-called “poor mobilizer” condition.

The present invention further relates to a method and kit for helping in the diagnosis of conditions of inflammation and/or impaired or absent marrow stem cell mobilization from the bone marrow to peripheral blood (“poor mobilizer”).

From the experiments described above it is clear that the adhesion protein CD169 is specific for classically activated macrophages, called also as proinflammatory M1 or M(LPS+IFNγ): the evaluation of gene and/or protein expression of CD169 therefore allows classically activated macrophages or proinflammatory macrophages to be detected on cells taken from peripheral blood or sections of tissue of bone marrow or marrow aspirate by Western Blot, flow cytometry, immunofluorescence or the like.

Said method and said kit comprise, for the evaluation of the protein expression, the in vitro use of an anti-CD169 antibody for the qualitative and quantitative identification of macrophages that express the protein CD169.

Anti-CD169 permits to label classically activated macrophages or proinflammatory macrophages M1 or M(LPS+IFNγ).

Marrow stem cells can be taken from the bone marrow in the region of bilateral posterior iliac crest of the patient and from the peripheral blood by apheresis.

The method for the prevention or the treatment of patients affected by diabetes and/or cardiovascular diseases and/or patients suffering from impaired or absent marrow stem cell mobilization from the bone marrow to peripheral blood, so-called “poor mobilizer” condition, comprises one or more of the following steps:

a) stimulation of the mobilization of marrow stem cells or progenitor cells from the bone marrow to peripheral blood;

b) collection of said cells from peripheral blood through apheresis or similar;

c) administration of said cells to the patient through infusion and/or injection and/or transplantation;

said step of stimulation being achieved through the administration of a pharmaceutical composition comprising at least one therapeutic agent that inhibits the production and/or the action of the human cytokine oncostatin M (OSM) as described above.

According to the present invention after the mobilization step, the cells can be collected from the peripheral blood and used for autologous or allogenic transplantation purposes or for administration purposes for prevention or treatment of one or more of the following diseases:

cardiovascular diseases,

hematologic diseases,

oncologic diseases,

nephrologic diseases,

metabolic diseases,

autoimmune diseases,

rheumatic diseases,

inflammatory diseases,

neurodegenerative diseases.

The mobilization method by inhibition of oncostatin M can be provided in combination with chemotherapeutic treatments, and/or administration of growth factors, such as G-CSF (granulocyte-colony stimulating factor), GM-CSF (granulocyte macrophage colony stimulating factor), and/or chemokines (for example SDF-la/CXCL12).

For example it is possible to provide to use known drugs such as drugs based on hemopoietic growing factors among which the GM-CSF (granulocyte macrophage colony stimulating factor) and G-CSF (granulocyte-colony stimulating factor).

Recombinant human GM-CSF and G-CSF or variants thereof can be used: for example it is possible to use a glycosylated and non-glycosilated, pegylated and non-pegylated growth factor, such as filgrastim, lenograstim (glycosylated G-CSF) and peg-filgrastim (pegylated G-CSF).

According to the present invention said at least one therapeutic agent is administered orally and/or parenterally (subcutaneously, intramuscular or intravenous).

According to one embodiment said at least one therapeutic agent is administered through carriers able to optimize delivery to the target organ or cell, such as liposomes, exosomes, micelles, microparticles, nanoparticles, nanostructured carriers or a combination of said carriers.

Therefore the process of autologous or allogenic transplantation or infusion of marrow stem cells can be divided into several steps:

administration of the pharmaceutical composition of the present invention, provided as an alternative or in combination with the use of other drugs to obtain cell mobilization, to stimulate the mobilization of stem cells from the bone marrow to the peripheral blood,

mobilization,

collecting cells for example by means of apheresis,

preparation of the product for a possible storage,

possible cryopreservation,

transplantation/infusion of marrow stem cells previously collected.

Considering that in the hematologic field some pathologies or some therapeutic treatments lead to cell toxicity and/or pancytopenia, a therapy supported by the infusion of marrow stem cells allows the normal hematopoiesis to be reconstructed, therefore allowing the patient to recover or to get better or to receive further antitumor therapies.

Considering also that the stem cell mobilization, associated or not to re-infusion and/or injection thereof at the level of ischemic tissues, previously has shown potential benefits in affected patients, a therapy able to stimulate more efficaciously the stem cell mobilization in diabetic patients and/or patients suffering from “poor mobilizer” condition allows higher effects of cardiovascular protection to be obtained.

Such process therefore is particularly suitable for the treatment of patients suffering from diabetes or from a “poor mobilizer” condition, to make auto-transplantations or to obtain cardiovascular protection.

Moreover it is suitable in order to obtain marrow stem cells by donors from peripheral blood.

Obviously it is possible to provide the composition, the treatment method and the method and kit for helping in the diagnosis according to the present invention to be applicable not only on the human body but also on the animal body it being possible to provide a pharmaceutical composition that comprises at least one therapeutic agent that inhibits the production and/or action of an animal oncostatin M (OSM) having functionality equivalent to the human oncostatin.

Variants and/or changes may be made to the composition and/or treatment method and/or method and kit for helping in the diagnosis according to the present invention without for this reason departing from the scope of protection claimed below.

### BIBLIOGRAPHIC REFERENCES

Bakanay S M et al. Bone Marrow Transplant, 2012; 47:1154-1163
Chow A et al. J Exp Med, 2011; 208:261-271
D'Rozario J et al. Transfus Apher Sci, 2014; 50:443-450
Fadini G P. Diabetologia, 2014; 57:4-15
Fadini G P et al. Atherosclerosis, 2010; 209:10-17
Fadini G P et al. Diabetes Care, 2013; 36:943-949
Fadini G P et al. Int J Cardiol, 2013; 168:892-897
Fadini G P et al. Diabetologia, 2013; 56:1856-1866
Fadini G P et al. PLoS One, 2010; 5: e11488
Fadini G P et al. Diabetologia, 2006; 49:3075-3084
Ferraro F et al. Sci Transl Med, 2011; 3: 104ra101
Guihard P et al. Stem Cells, 2012; 30:762-772
Lapidot T et al. Exp Hematol, 2002; 30:973-981
Lee M J et al. Int J Biochem Cell Biol, 2007; 39:650-659
Leone A M et al. Eur Heart J, 2005; 26:1196-1204 Levesque J P et al. J Clin Invest, 2003; 111:187-196 Ling L et al. PLoS One, 2012; 7: e50739
Minehata K et al. Int J Hematol, 2006; 84:319-327 Moazzami K et al. Cochrane Database Syst Rev, 2013; 5:CD008844
Pillarisetti S. Cardiovasc Hematol Agents Med
Chem, 2012; 10:185-189
Seshasai S R et al. N Engl J Med, 2011; 364:829-841 Van Rooijen N et al. J Immunol Methods,
1994; 174:83-93
Zimmet H et al. Eur J Heart Fail, 2012; 14:91-105

The invention claimed is:

1. A pharmaceutical composition to induce bone marrow stem cell mobilization from bone marrow to peripheral blood during treatment of cardiovascular diseases, or in patients subjected to treatments that impair cell mobilization, or in patients suffering from a “poor mobilizer” condition, comprising:
at least one therapeutic agent that inhibits production and/or action of a human cytokine oncostatin M (OSM), a macrophage derived factor that prevents mobilization of stem cells from the bone marrow to peripheral blood.

2. The pharmaceutical composition according to claim 1, wherein said at least one therapeutic agent carries out one or more of the following functions, provided in combination or alternatively to one another:
a) inhibition of oncostatin M (OSM) production by cells;
b) neutralization and/or blocking of extracellular oncostatin M (OSM);
c) inhibition and/or neutralization of oncostatin M (OSM) receptor or a subunit thereof;
d) inhibition of binding of the oncostatin M (OSM) to its receptor; or
e) inhibition and/or blocking of intracellular effects of activation of the receptor by the oncostatin M (OSM).

3. The pharmaceutical composition according to claim 1, wherein said therapeutic agent is at least a monoclonal antibody specifically directed against the oncostatin M (OSM).

4. The pharmaceutical composition according to claim 1, wherein said therapeutic agent is at least a monoclonal antibody specifically directed against an oncostatin M (OSM) receptor or a subunit of said receptor.

5. The pharmaceutical composition according to claim 1, wherein said therapeutic agent is at least a monoclonal antibody specifically directed against an OSMR (oncostatin M receptor), gp130 (interleukin-6 family receptor), or OSMR/gp130 heterodimer.

6. The pharmaceutical composition according to claim 1, wherein said at least one therapeutic agent is a compound or mixture of compounds selected from the group consisting of a binding protein, a soluble receptor, a degrading enzyme, a neutralizing antibody, a blocking monoclonal antibody or a fragment thereof, or an anti-sense RNA, said compound or a mixture of compounds inhibiting or neutralizing an oncostatin M protein, by sequestering and/or degrading the oncostatin M (OSM) and/or preventing the oncostatin M (OSM) from binding to its receptor.

7. The pharmaceutical composition according to claim 1, wherein said at least one therapeutic agent is a compound or a mixture of compounds inhibiting oncostatin M (OSM) production at cellular level and selected from the group consisting of an enzyme inducer, an enzyme or receptor inhibitor, a ligand of a receptor in the cell surface or cytoplasm or nucleus, a compound that is toxic for the cells, or an antisense RNA.

8. The pharmaceutical composition according to claim 1, wherein said at least one therapeutic agent is a compound or a mixture of compounds inhibiting or neutralizing an oncostatin M (OSM) receptor or a subunit of said receptor and selected from the group consisting of a chemical inhibitor or antagonist or partial agonist of said receptor, a degrading enzyme, an antibody blocking said receptor, a monoclonal antibody blocking said receptor, a fragment of a monoclonal antibody directed against said receptor, an antisense RNA directed against messenger RNA of a receptor gene, or any agent that prevents oncostatin M (OSM) from eliciting its biological effects through binding to its receptor.

9. The pharmaceutical composition according to claim 1, wherein said at least one therapeutic agent is a compound or a mixture of compounds inhibiting biological effects of the oncostatin M (OSM) in mesenchymal stromal stem cells and selected from the group consisting of a chemical compound that targets a molecule of an intracellular transduction signalling cascade elicited by a binding of the oncostatin M (OSM) to its receptor, a chemical compound which is a receptor or enzyme inducer or inhibitor, a ligand of a receptor in cell surface, a cytoplasm and/or nucleus, or an antisense RNA.

10. The pharmaceutical composition according to claim 1, wherein the pharmaceutical composition comprises, as the therapeutic agent, one or more gene silencers selected from the group consisting of single-stranded RNA synthetic oligonucleotides (antisense RNA) and/or double-stranded RNA complementary to mRNA (messenger RNA) encoding for the oncostatin M (OSM), said gene silencers being comprised in combination or alternatively to one another, such to obtain silencing that causes a decreased expression of gene encoding for the oncostatin M (OSM).

11. The pharmaceutical composition according to claim 1, wherein the pharmaceutical composition comprises, as the therapeutic agent, one or more gene silencers selected from the group consisting of single-stranded RNA synthetic oligonucleotides (antisense RNA) and/or double-stranded RNA complementary to mRNA (messenger RNA) encoding for the oncostatin M (OSM) receptor or a subunit thereof, said gene silencers being comprised in combination or alternatively to one another, such to obtain silencing that causes a decreased expression of gene encoding for an oncostatin M receptor or a subunit of said receptor.

12. The pharmaceutical composition according to claim 1, further comprising a vector for said at least one therapeutic agent that is not part of the at least one therapeutic agent, said vector optimizing delivery to a target organ or cell.

13. The pharmaceutical composition according to claim 1, said at least one therapeutic agent is provided in combination with one or more chemotherapies and/or growth factors, which are not an integral part of the pharmaceutical composition.

14. The pharmaceutical composition according to claim 1, wherein said at least one therapeutic agent is provided in combination with at least one pharmacologically acceptable excipient for treatment of pathological conditions of impaired cell mobilization from bone marrow to peripheral blood, and/or treatment of phenomena associated thereto, said at least one excipient being not an integral part of the pharmaceutical composition.

15.-20. (canceled)

21. A method of preventing, or treating a patient affected by, diabetes, cardiovascular diseases, or impaired or absent marrow stem cell mobilization from bone marrow to peripheral blood, comprising one or more of the following steps:
a) stimulating mobilization of cells that include marrow stem cells or progenitor cells from the bone marrow to the peripheral blood;
b) collecting said cells from the peripheral blood; and
c) administering said cells to the patient through infusion, injection, or transplantation,
wherein said step of stimulating comprises administering a pharmaceutical composition comprising at least one therapeutic agent that inhibits production or action of human cytokine oncostatin M (OSM) according to claim 1.

22. The method according to claim 21, further comprising the step of administering chemotherapeutic treatments, growth factors, or chemokines.

23. The method according to claim 21, wherein said at least one therapeutic agent is administered orally or parenterally.

24. The method according to claim 21, wherein said at least one therapeutic agent is administered through one or more carriers that optimize delivery to a target organ or cell, said carriers comprising liposomes, exosomes, micelles, microparticles, nanoparticles, or nanostructured carriers, said carriers being provided alone or combined to each other.

25. (canceled)
